(19) United States
(12) Patent Application Publication
(10) Pub. No.: US 2007/0299490 A1
(43) Pub. Date: Dec. 27, 2007

(54) RADIOFREQUENCY (RF)-SHUNTED SLEEVE HEAD AND USE IN ELECTRICAL STIMULATION LEADS

(76) Inventors: Zhongping Yang, Woodbury, MN (US); John L. Sommer, Coon Rapids, MN (US); James D. Reinke, Maple Grove, MN (US)

Correspondence Address: MEDTRONIC, INC., 710 MEDTRONIC PARKWAY NE, MINNEAPOLIS, MN, 55432-9924, US

(21) Appl. No.: 11/555,111

(22) Filed: Oct. 31, 2006

Related U.S. Application Data
(63) Continuation-in-part of application No. 11/426,207, filed on Jun. 23, 2006.
(60) Provisional application No. 60/826,476, filed on Sep. 21, 2006.

(51) Int. Cl. A61N 1/16

(52) U.S. Cl. 607/116; 607/119

(57) ABSTRACT

A medical device lead is presented. The medical device lead includes a lead body and a tip electrode. A radio frequency shunted sleeve head is coupled to the lead body and to the tip electrode. The sleeve head includes a proximal end and a distal end. A biostable dielectric coating is introduced over the sleeve head.

### RELATED APPLICATIONS

This application is a continuation-in-part of U.S. patent application Ser. No. 11/426,207 filed on Jun. 23, 2006, entitled “ELECTRODE SYSTEM WITH SHUNT ELECTRODE”, to Volkert A. Zeijlemaker, incorporated herein by reference in its entirety. This application also claims priority to U.S. Provisional Application Ser. No. 60/826,476, filed Sep. 21, 2006

### TECHNICAL FIELD

The invention relates to medical devices and, more particularly, to implantable medical device leads for use with implantable medical devices (IMDs).

### BACKGROUND

In the medical field, implantable leads are used with a wide variety of medical devices. For example, implantable leads are commonly used to form part of implantable cardiac pacemakers that provide therapeutic stimulation to the heart by delivering pacing, cardioversion or defibrillation pulses. The pulses can be delivered to the heart via electrodes disposed on the leads, e.g., typically near distal ends of the leads. In that case, the leads may position the electrodes with respect to various cardiac locations so that the pacemaker can deliver pulses to the appropriate locations. Leads are also used for sensing purposes, or for both sensing and stimulation purposes. Implantable leads are also used in neurological devices, muscular stimulation therapy, and devices that sense chemical conditions in a patient's blood, gastric system stimulators.

Occasionally, patients that have implantable leads may benefit from a magnet resonance image being taken of a particular area of his or her body. Magnetic resonance imaging (MRI) techniques achieve a more effective image of the soft tissues of the heart and vascular system. MRI procedures can also image these features without delivering a high dosage of radiation to the body of the patient, and as a result, MRI procedures may be repeated reliably and safely. However, MRI devices may operate at frequencies of 10 megahertz or higher, which may cause energy to be transferred to the lead. In particular, the high frequency fields induce a voltage in the lead, causing the potential of the lead to be higher than the surrounding tissue. In effect, the lead behaves as an antenna. Current may flow from the electrode into the tissue proximate to the electrode due to induced voltage. It is therefore desirable to develop a lead addresses this disadvantage.

### BRIEF DESCRIPTION OF DRAWINGS

Aspects and features of the present invention will be appreciated as the same becomes better understood by reference to the following detailed description of the embodiments of the invention when considered in connection with the accompanying drawings, wherein:

FIG. 1 is a conceptual perspective view of a medical device system including a medical device coupled to a lead according to an embodiment of the present invention;

FIG. 2 is a cross-sectional view of an electrode assembly located at a distal end of a medical lead;

FIG. 3 depicts multiple layers of insulating material over a conductive element of the electrode assembly depicted in FIG. 2;

FIG. 4A depicts a cross-sectional view of a conductive ring coupled to a conductive sealer for the electrode assembly depicted in FIG. 2;

FIG. 4B depicts a top view of a conductive ring coupled to a conductive sealer for the electrode assembly depicted in FIG. 2;

FIG. 4C depicts a cross-sectional view of conductive rings and a conductive sealer coupled to a shaft;

FIG. 4D depicts an angled view of a conductive sealer;

FIG. 5 is a schematic diagram of a simplified circuit for a medical device system under pacing and sensing conditions;

FIG. 6 is a schematic diagram of another simplified circuit for a medical device system under magnetic resonance imaging conditions;

FIG. 7 is a block diagram of an electrochemical impedance spectrum (EIS) measurement system;

FIG. 8 graphically depicts an EIS measurement which shows an impedance spectrum and the frequency corresponding to typical pacing conditions;

FIG. 9 graphically depicts reduced heating in the tip of the medical lead; and

FIG. 10 is a flow diagram that depicts the method of producing an electrode assembly.

### DETAILED DESCRIPTION

The present invention is directed to a medical lead, techniques for manufacturing such a lead, and systems that include a medical device coupled to a medical lead according to the present invention. The medical lead of the present invention includes a radio frequency signal (RF) shunted sleeve head (also referred to as a capacitive shunt). The RF shunted sleeve head is coupled to a lead body and to the tip electrode. The RF shunted sleeve head comprises a biostable dielectric coating introduced over the conductive element.

The medical lead is able to effectively manage high frequency signals from other devices such that the operation of a medical lead is not detrimentally affected. For example, the electrode assembly of the medical lead shunts the high frequency RF signals (e.g. 21 megaHertz (Mhz) to 128 MHz) generated from a magnetic resonance imaging (MRI) machine away from the tip electrode and into the larger area of the RF shunted sleeve head. This in turn, reduces the current density in the tissue near the electrode and reduces the level of heating. Consequently, a patient with a medical lead may undergo an MRI procedure without significantly affecting the operation of the medical lead.

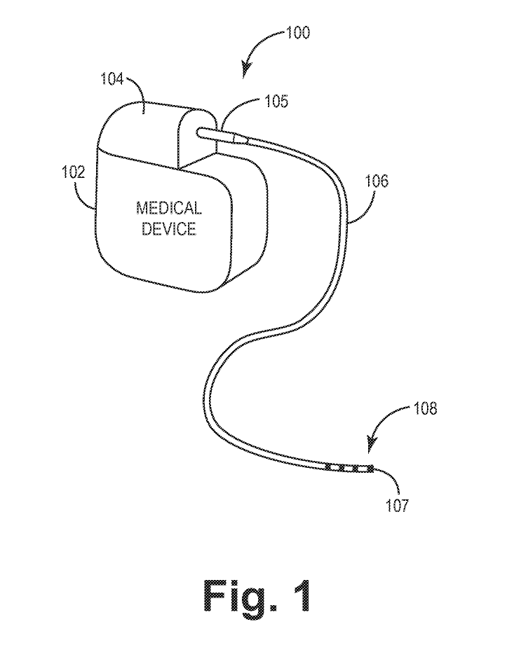

FIG. 1 depicts a medical device system 100. A medical device system 100 includes a medical device housing 102 having a connector module 104 that electrically couples various internal electrical components of medical device housing 102 to a proximal end 105 of a medical lead 106 (also referred to as a MRI/RF shunted lead, or a shunted lead). A medical device system 100 may comprise any of a wide variety of medical devices that include one or more medical lead(s) 106 and circuitry coupled to the medical lead(s) 106. An exemplary medical device system 100 may take the form of an implantable cardiac pacemaker, an implantable cardioverter, an implantable defibrillator, an implantable cardiac pacemaker-cardioverter-defibrillator (PCD), a neurostimulator, or a muscle stimulator. Medical device system 100 may deliver, for example, pacing, cardioversion or defibrillation pulses to a patient via electrodes 108 disposed on distal ends 107 of one or more lead(s) 106. In other words, lead 106 may position one or more electrodes 108 with respect to various cardiac locations so that medical device system 100 can deliver pulses to the appropriate locations.

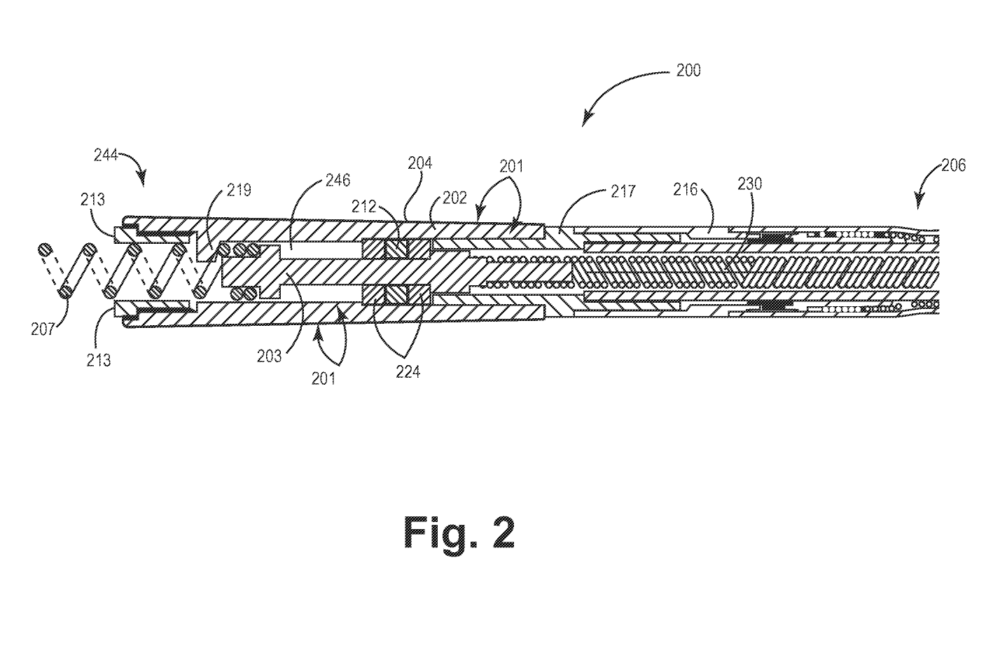

FIG. 2 depicts an electrode assembly 200 of a medical lead 106. Electrode assembly 200 includes a RF-shunted sleeve head 201 coupled to an electrode 207 (also referred to as a tip electrode), a monolithic controlled-release device (MCRD) 213, a conductive electrode shaft 203, a conductive sealer 212, conductive rings 224, a ring electrode 216, and a non-conductive spacer 217. At a distal end 244 of electrode assembly 200, a sharpened distal tip (not shown) facilitates fixation of the distal end of helically shaped electrode 207 into tissue of a patient. The proximal end of electrode 207 is securely seated between MCRD 213, electrode shaft 203, and a securing member 219 that protrudes from an inner diameter of RF shunted sleeve head 201. MCRD 213 provides chronic steroid elution to maintain a low pacing threshold for a medical device system 100.

RF-shunted sleeve head 201 is electrically connected to a conductive electrode shaft 203 via two parallel conductive rings 224 (e.g. C-rings etc.) and a conductive sealer 212 (also referred to as a sealing washer). At a proximal end 206 of electrode assembly 200, coil 230 is electrically coupled to conductive electrode shaft 203. RF shunted sleeve head 201 comprises a conductive element 202 surrounded or at least partially covered by an insulating material 204 (also referred to as a dielectric material). In one embodiment, conductive element 202 is cylindrically shaped (e.g. ring, etc.) or may possess other suitable shapes. Exemplary dimensions for conductive element 202 include a diameter of about 6.5 French (Fr.) by about 9 millimeters (mm) in length, an outer diameter of about 82 mils and an inner diameter of about 62 mils. Conductive element 202, in one embodiment, includes an increased diameter at the distal end and a reduced diameter at the proximal end of the conductive element 202. The surface area of conductive element 202 is about 60 mm² which is much larger than the 5.5 mm² surface area of electrode 207. A large surface area ratio, defined by the ratio of the surface area of conductive element 202 to the surface area of electrode 207, is desired to insure that current induced from a MRI machine is spread over a large area of electrode assembly 200. A tenfold (i.e. 10×) larger surface area ratio results in about tenfold lower temperatures at the tip of electrode 207 assuming ring electrode 216 has low impedance at high frequencies. Conductive element 202 comprises materials that are chemically stable, biocompatible, and x-ray transparent. Exemplary material used to form conductive element 202 includes titanium, titanium alloy, conductive polymers, and/or other suitable materials.

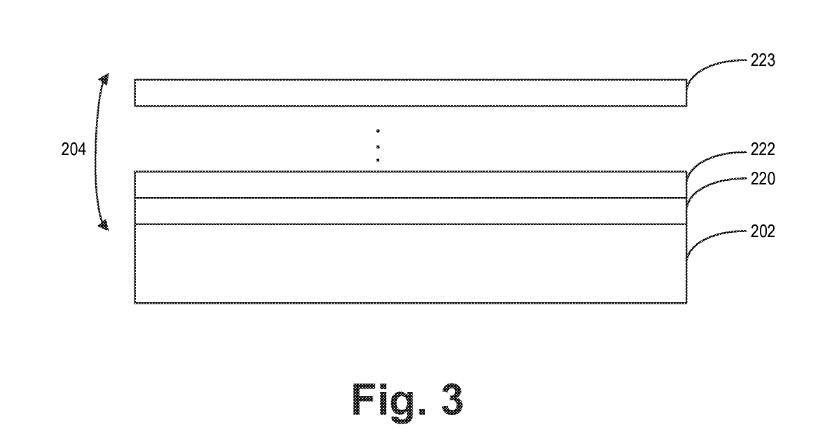

Referring to FIG. 3, insulative material 204 may be formed from a single layer or multiple layers such as first layer 220, second layer 222, and N layer 223, where N is a whole number that is less than 100, and is typically less than about 30 layers. Each layer may comprise different insulating materials, two or more different insulating materials, or the same insulating materials. Insulative material 204 includes a thickness from about 1 nanometer (nm) to about 1 millimeter (mm)) and extends from about 1 mm to about 20 mm along the length of conductive element 202. Insulative material 204 may be formed from any of a wide variety of insulating materials. Exemplary insulating material include at least one or more of Parylene, polyamide, metal oxides, polyimide, urethane, silicone, tetrafluroethylene (ETFE), polytetrafluroethylene (PTFE), or the like. Parylene is the preferred insulating material 204. The preferred Parylene is Parylene C. Parylene C is formed through a dimer vacuum deposition process. The dimer is commercially available from Specialty Coating Systems located in Clear Lake, Wis. Numerous techniques may be employed to introduce insulating material 204 over the outside of sleeve head 201 and/or partially inside sleeve head 201. Exemplary techniques include chemical vapor deposition, dip coating, or thermal extrusion.

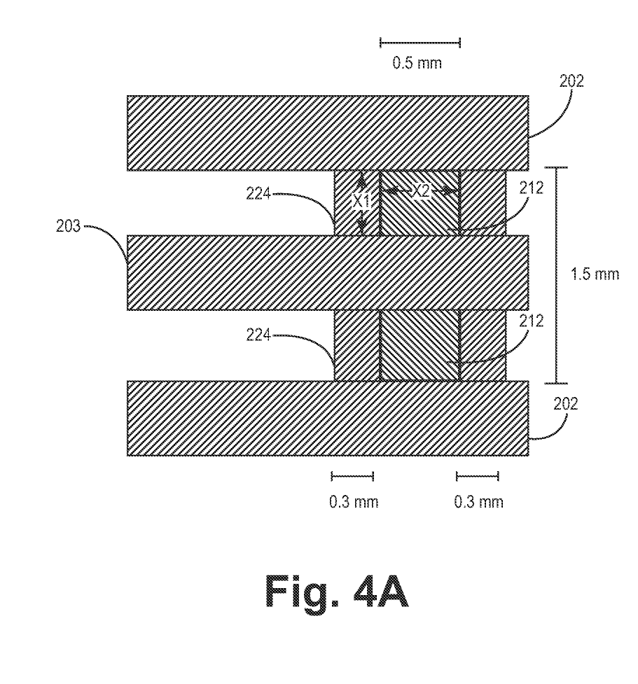

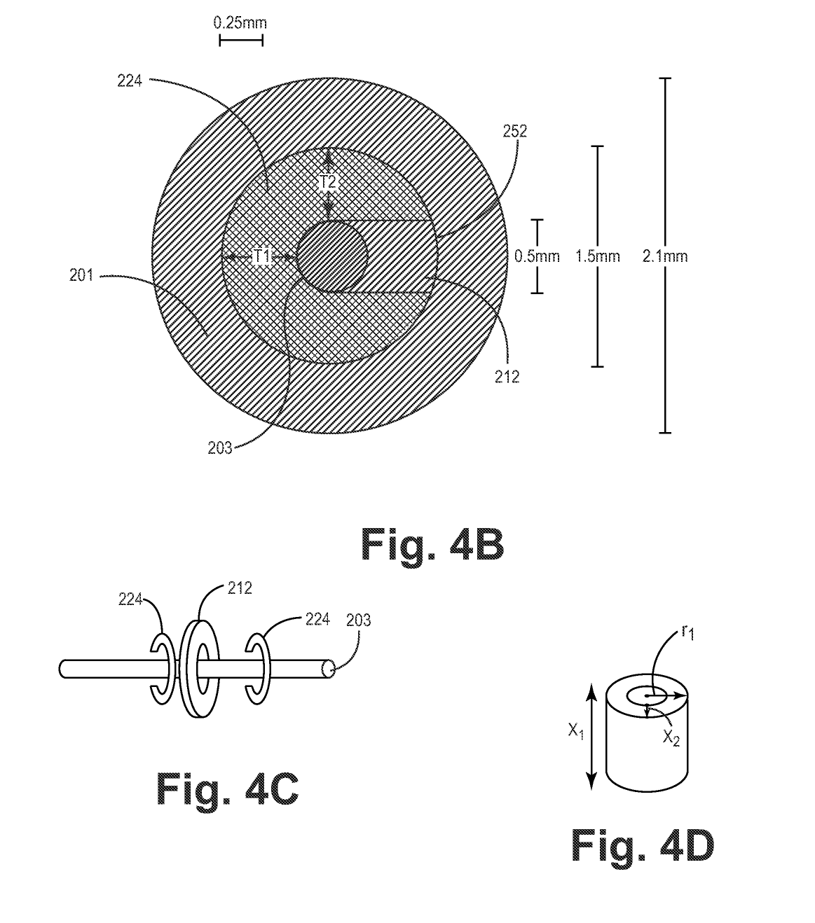

Conductive sealer 212 conducts current and also prevents fluid from passing through lumen 246. Referring to FIGS. 4A-4D, conductive sealer 212 is substantially ring (i.e. o-ring) or disk shaped but other suitable shapes may also be employed. In one embodiment, conductive sealer 212 is defined by X1, X2 and radius (r1). X1 ranges from about 0.1 mm to about 0.50 mm, X2 extends from about 0.1 mm to about 1.0 mm, and r1 extends from about 0.5 mm to about 1.0 mm. Curved end 252 extends to about 1.25 mm from the center of shaft 203 and includes a curve defined by a radius of about 0.5 mm.

Conductive sealer 212 comprises a polymer and a conductive polymer such as a conductive powder (e.g. carbon, carbon nanotube, silver, platinum etc.). The conductive polymer ranges from about 1% to about 25% of conductive sealer 212. The polymer (e.g. silicone etc.) is commercially available from Nusil Technology LLC, located in Carpinteria, Calif. Polyurethane is commercially available from The Polymer Technology Group Inc. located in Berkeley, Calif.

Conductive rings 224 are shaped, in one embodiment, as a C-ring to receive conductive sealer 212. Conductive rings 224 have an outer diameter of about 1.5 mm, an inner diameter of about 0.7 mm, and a thickness that ranges from about 0.25 mm (T1) to about 0.5 mm (T2). Conductive rings 224 are comprised of platinum or other suitable materials.

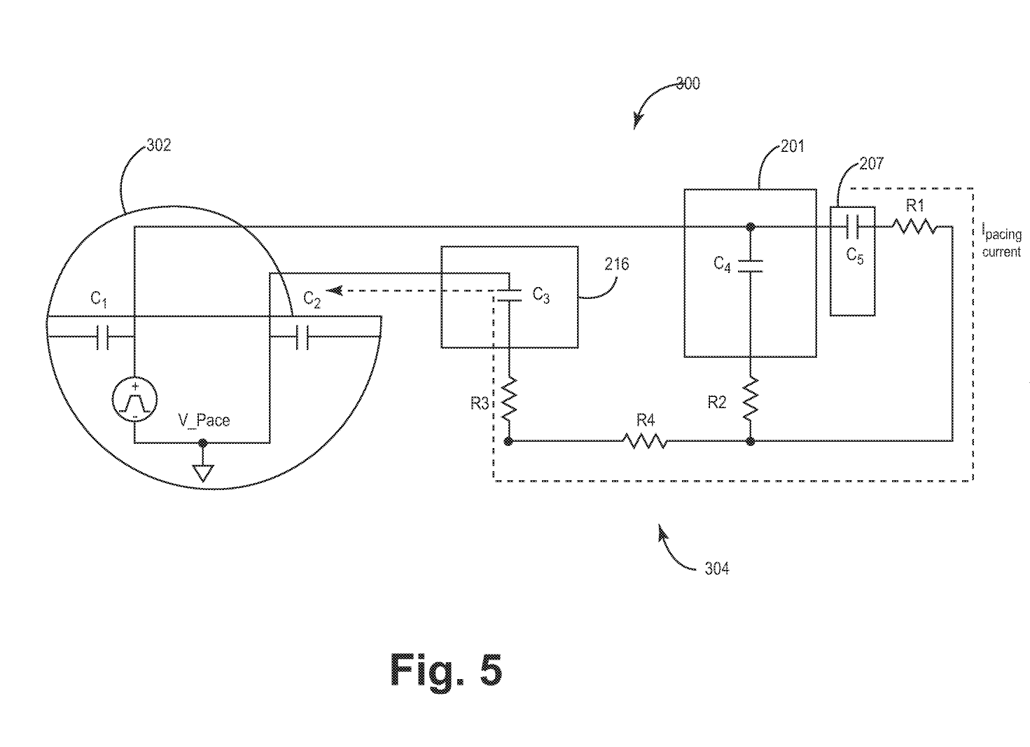

FIG. 5 depicts a simplified circuit 300 for a medical device system 100 during normal pacing conditions. Pacing conditions typically involve low frequency signals (e.g. 1000 Hz). Circuit 300 includes an implantable medical device (IMD) circuit 302 (e.g. a pacemaker circuit, neurostimilator circuit etc.) connected to a bipolar shunted lead circuit 304. IMD circuit 302 comprises two filter capacitors C1 and C2 connected to housing 102. C1 and C2 filter high frequency electromagnetic interference (EMI) so that high frequency signals from a MRI machine do not affect the sensing operation of medical lead 106. Exemplary values for C1 is about 1 to 10 nanoFarad (nF) and C2 is 1-10 nF.

Bipolar shunted lead circuit 304 includes ring electrode 216, RF shunted sleeve head 201, and tip electrode 207. Capacitors C3, C4, and C5 correspond to ring electrode 216, sleeve head 201, and tip electrode 207, respectively. Resistors R1, R2, R3, and R4 represent the impedance created by tissue and/or blood of the patient. R1, R2, and R3 along with capacitors C5, C4, and C3 represent the electrode to tissue interface impedances. Generally, larger area electrodes result in larger values of capacitance and smaller values of resistance. However, the addition of an insulating material 204 over sleeve head 201 reduces the effect of electrode 207 to tissue interface impedances and its capacitance. In particular, C4 comprises a series capacitance of the electrode to tissue interface and capacitance due to insulation. Exemplary values for bipolar shunted lead circuit 304 include C3 at 10 microF (uF), R3 is 100 Ohm (Ω), R2 is 100Ω, C5 is 1 uF, R1 is 500Ω, and C4 is about 0.5 nanoFarad (nF) to about 10 nF. Optimally, C4 should possess a capacitance of about 1-2 nF.

Generally, under typical pacing conditions, pacing current (I_{pacing current}) flows from tip electrode 207 to ring electrode 216 and then returns to IMD circuit 302. Negligible or no current I_{pacing current} passes through the RF shunted sleeve head 201 and resistor R2 because under a low frequency or direct current (DC) application, capacitor C4 acts like an open circuit to a constant voltage across its terminals. A portion of the I_{pacing current} passes to the patient's tissue, represented as resistor R1, due to the large capacitance of C5 associated with tip electrode 207. Similarly, a portion of the I_{pacing current} passes to the patient's tissue, represented as resistor R3, due to the large capacitance of C3 associated with ring electrode 216.

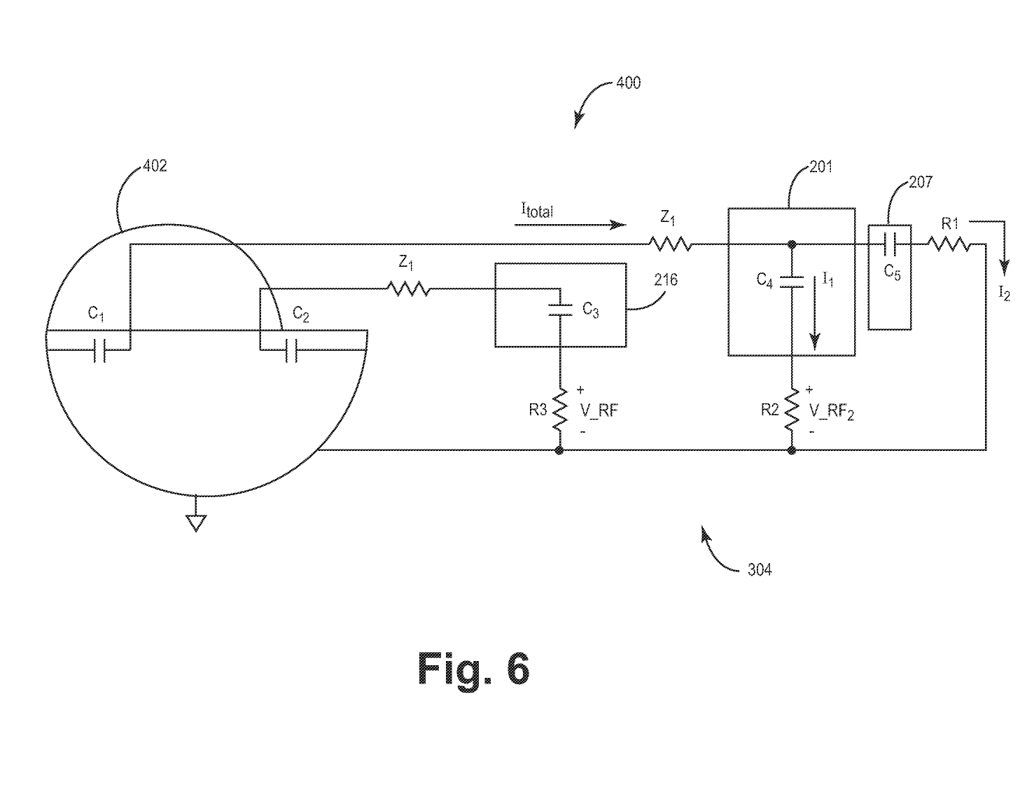

FIG. 6 depicts a simplified circuit 400 for a medical device system 100 during MRI conditions. Circuit 400 includes an IMD circuit 402 (e.g. a pacemaker circuit, neurostimulator circuit etc.) and a bipolar shunted lead 304. Circuit 400 includes the same elements as circuit 300, except lead resistance Z₁ is depicted between IMD circuit 402 and ring electrode 216 and also between IMD circuit 402 and RF shunted sleeve 201. Additionally, voltage potential differentiators (i.e. V-RF, V-RF2), are induced from a RF field.

Under MRI conditions, a large current (Iₜₒₜₐₗ) is induced in the medical lead 106 and IMD circuit 402 due to the RF voltage sources VRF and VRF2. A portion of the current, I₁, passes through the RF shunt sleeve head 201 represented by R2 and C4. Since the RF frequency is large, the impedance associated with C4 is small resulting in a large portion of the total current flowing through RF shunt sleeve head 201. The remainder of the current, 12, passes through tip electrode 207, represented by capacitor C5. The current then returns to capacitor C2 of IMD circuit 402 through ring electrode 216 and through the body tissue and housing 102. Because conductive element 202 of RF shunted sleeve head 201 has a large surface area relative to tip electrode 207, the total current is spread over a larger total surface area resulting in a lower current density. This results in reduced local heating at the tip of electrode 207. In sum, RF shunted sleeve head 201 and tip electrode 207 cooperate to serve as a high-pass filter, allowing only high frequencies signals to “pass” through conductive element 202 and low frequency signals are blocked. RF shunted sleeve head 201 serves as a capacitor and passes the MRI high frequency current into the blood stream (represented by R2 in FIG. 6) surrounding sleeve head 201 rather than through tip electrode 207 and into the myocardium tissue (represented by R1).

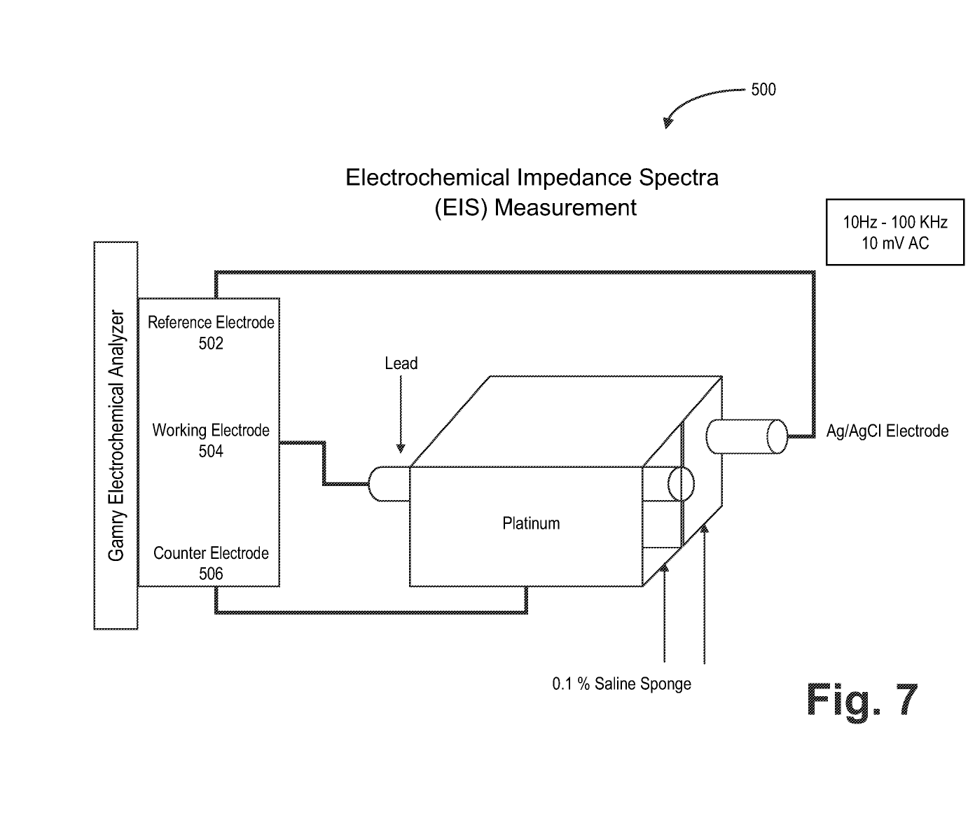

FIG. 7 is a block diagram of an electrochemical impedance spectrum (EIS) testing system 500 that assists in determining the optimal RF shunted sleeve head 201 for a medical lead 106. In particular, testing system 500 evaluates the electrochemical properties associated with RF shunted sleeve head 201. These electrochemical properties depend upon material(s) from which conductive element 202 and insulating material 204 are made. Testing system 500 includes a reference electrode 502, a tip electrode 504, and a counter electrode 506. RF shunted sleeve head 201 is evaluated by being inserted into a 0.1% saline soaked sponge located in a platinum container. Tip electrode 207 is not in the saline while ring electrode 216 is in the saline solution. Reference electrode 502, comprised of Ag/AgCl, is connected to the saline sponge. The performance of RF shunted sleeve head 201 is determined while it is exposed to signals at various frequencies between 100000 Hz and 0.01 HZ. Testing system 500 was used to optimize RF shunted sleeve head 201 for a medical lead 106.

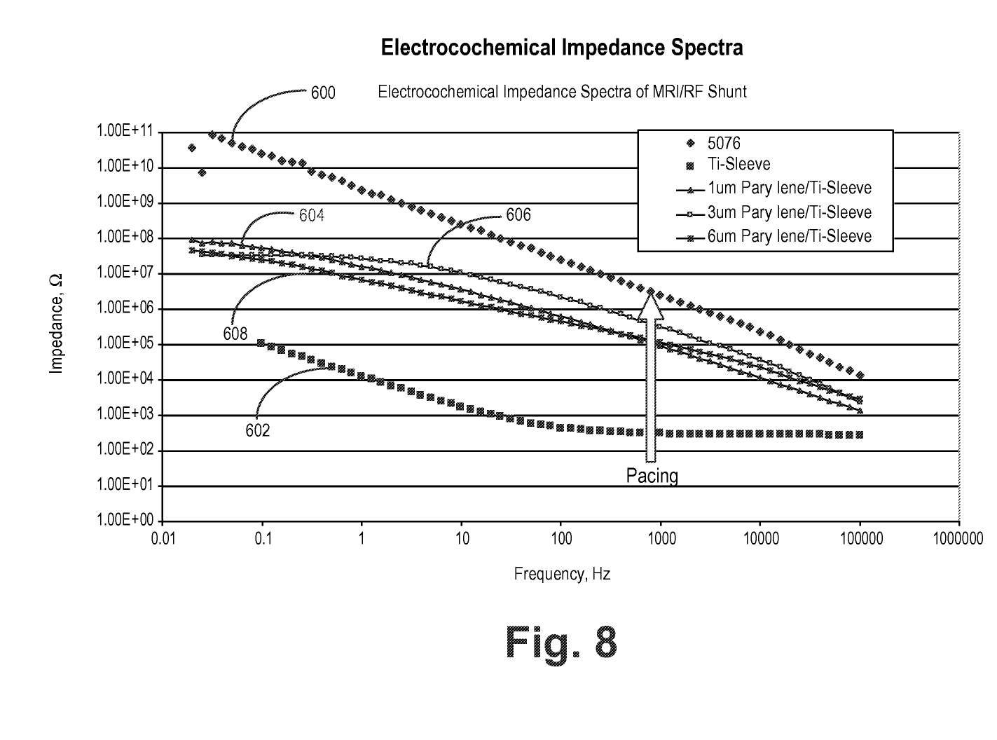

FIG. 8 graphically confirms that the RF shunted sleeve head 201 is able to achieve the desired capacitance that will result in heat reduction at electrode 207. The curves represent the impedance measured from tip electrode 207 conductor of lead 106 to the saline soaked sponge with tip electrode 207 out of the saline solution, but ring electrode 216 in the solution. RF shunted sleeve head 201 was exposed to signals of about 0.01 KHz to about 100 KHz. A line 600 represents the impedance of sleeve head 201 on a standard lead. The impedance is high (e.g. 2.5 mega Ω) at typical pacing frequencies. The measured impedance is dominated by the capacitance from tip electrode 207 conductor to the ring electrode conductor due to the coaxial lead construction. A line 602 is the impedance of a bare titanium RF shunted sleeve head 201 with no insulating material 204. The impedance is very low, especially at high frequencies. Lines 604, 606, and 608, respectively represent the impedance of the different capacitive shunt which vary depending on the thickness of the parylene coating. The impedance is high at pacing and sensing frequencies (over 100 kΩ). In general, thicker layers of insulating material 204 are expected to provide higher values of impedance. The discrepancy between the observed data and the predicted data can be caused by variations in the quality and consistency of insulating material 204 (e.g. Parylene dielectric coating etc.). This is primarily evident at the low frequency where the capacitance plays less of a role in setting the impedance.

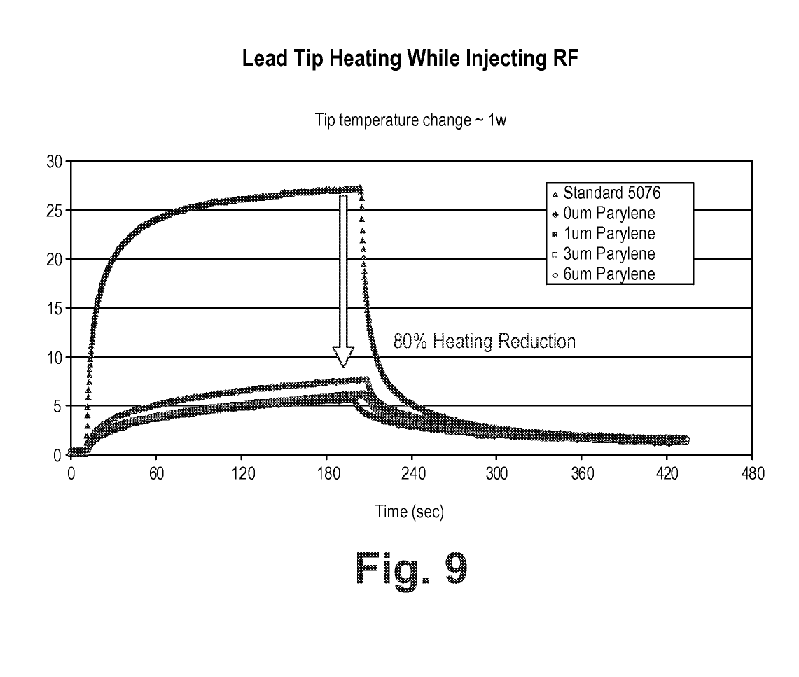

FIG. 9 graphically depicts that lead 106 is able to operate under MRI conditions. A significant drop in temperature rise exists with RF shunted sleeve head 201. For example, at least 80% of the RF power is shunted away from tip electrode 207 by sleeve head 201 which reduces the amount of heat at the electrode. Specifically, a standard lead exhibits peak heating at 27.0 Celcius (° C.) after 3 minutes. For a titanium shunted lead with no insulation, peak heating is 5.6° C. after 3 minutes. These results are achieved, in part, through a large effective surface area tip electrode at MRI frequencies (i.e. 21-128 megahertz (MHz) for 0.5-3T machines) and a normal effective surface area for pacing frequencies (i.e. 100 Hz-10 KHz) or sensing frequencies (i.e. 0.1-100 hz). The large electrode area at MRI frequencies (1) creates a low impedance which reflects some of the energy away from the tip electrode 207 and back up the lead body, and (2) spreads the power over a larger surface area decreasing the peak temperature.

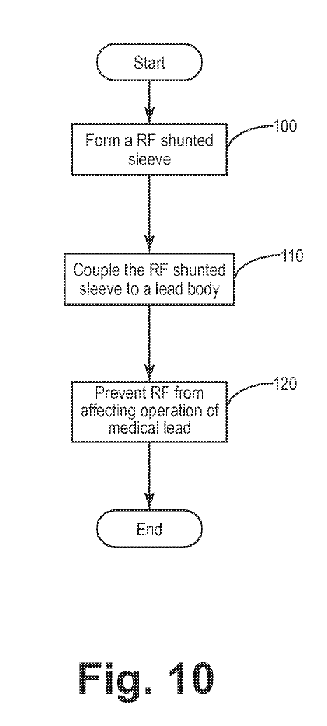

FIG. 10 is a flow diagram that depicts the method of producing a medical lead. At block 300, a RF shunted sleeve head is formed. The RF shunted sleeve head comprises a biostable dielectric coating introduced over the conductive element. At block 320, the RF shunted sleeve head is coupled to the lead body. At block 330, the RF is prevented from affecting the sensing operation of the medical lead.

It is understood that the present invention is not limited for use in pacemakers, cardioverters of defibrillators. Other uses of the leads described herein may include uses in patient monitoring devices, or devices that integrate monitoring and stimulation features. In those cases, the leads may include sensors disposed on distal ends of the respective lead for sensing patient conditions.

The leads described herein may be used with a neurological device such as a deep-brain stimulation device or a spinal cord stimulation device. In those cases, the leads may be stereotactically probed into the brain to position electrodes for deep brain stimulation, or into the spine for spinal stimulation. In other applications, the leads described herein may provide muscular stimulation therapy, gastric system stimulation, nerve stimulation, lower colon stimulation, drug or beneficial agent dispensing, recording or monitoring, gene therapy, or the like. In short, the leads described herein may find useful applications in a wide variety medical devices that implement leads and circuitry coupled to the leads.

Various embodiments of the invention have been described. These and other embodiments are within the scope of the following claims. For example, electrode 207 may include variously shaped electrodes such as ring shaped or other suitable shapes. Additionally, skilled artisans appreciate that other dimensions may be used for the mechanical and electrical elements described herein. Moreover, it is expected that 100% of the RF power is able to be shunted away by the RF sleeve head by implementing the claimed embodiment as well as other features.

1. A medical device lead comprising:
a lead body;
a tip electrode coupled to the lead body, and
a radio frequency (RF) shunted sleeve head coupled to the lead body.

2. The medical device lead of claim 1, wherein high frequency signals being shunted away from the tip electrode and into the sleeve head.

3. The medical device lead of claim 1, wherein the RF shunted sleeve head includes:
a conductive element having a proximal end and a distal end; and
a biostable dielectric coating introduced over the conductive element.

4. The medical device lead of claim 3, wherein a surface area of the conductive element being about ten times larger than a surface area of the tip electrode.

5. The medical device lead of claim 3, wherein the dielectric coating extends over an increased diameter at the distal end and a reduced diameter at the proximal end of the conductive element.

6. The medical device lead of claim 4, further comprising a conductive sealer coupled to the conductive element.

7. The medical device lead of claim 3, wherein the biostable dielectric coating being at least one of polyamide, parylene, and metal oxides.

8. The medical device lead of claim 3, wherein the conductive element being at least one of titanium, titanium alloy, and conductive polymers.

9. The medical device lead of claim 2, wherein at least 80% of RF power being shunted away by the RF sleeve head.

10. The medical device lead of claim 2, wherein at least 70% of RF power being shunted away by the RF sleeve head.

11. The medical device lead of claim 2, wherein at least 60% of RF power being shunted away by the RF sleeve head.

12. A medical device lead comprising:
a lead body;
a conductive element coupled to the lead body, the conductive element having a proximal end and a distal end; and
a biostable dielectric coating introduced over the conductive element which extends over an increased diameter at the distal end and a reduced diameter at the proximal end of the conductive element.

13. The medical device lead of claim 12, wherein the biostable dielectric coating being at least one of polyamide, parylene, and metal oxides.

14. The medical device lead of claim 12, further comprising:
a conductive sealer coupled to the conductive element.

15. The medical device lead of claim 13, wherein the conductive sealer comprising at least one of silicone carbon and silicone platinum.

16. A lead circuit for an implantable medical device comprising:
a first impedance and a first capacitor associated with an RF shunted sleeve head; and
a second capacitor associated with a tip electrode.

17. The lead circuit of claim 16, wherein the first capacitor has a capacitance that ranges from about 0.5 nanofarads (nF) to about 10 nF.

18. The lead circuit of claim 16 wherein a lead avoids detrimental affects associated with magnetic resonance imaging.

19. A method for forming a medical device lead comprising:
providing a conductive element, the conductive element having a proximal end and a distal end;
introducing multiple layers of biostable insulating material over the conductive element;
coupling a conductive sealer to the conductive element; and
coupling the conductive sealer and the conductive element to a lead body.

20. The method of claim 19 wherein introducing the dielectric coating being implemented by one of chemical vapor deposition, dip coating, and thermal extrusion.

21. The method of claim 19, wherein a surface area of the conductive element being about ten times larger than a surface area of a tip electrode, the tip electrode coupled to the conductive element.

22. The method of claim 19, wherein at least 80% of RF power being shunted away by the conductive element.

23. The method of claim 19, wherein the biostable insulating layer being at least one of polyamide, parylene, and metal oxides.

24. The method of claim 19, wherein the multiple layers of biostable insulating material comprises a first layer being at least one of polyamide, parylene, and metal oxides and a second layer being at least one of polyamide, parylene, and metal oxides, the second layer comprises a different insulating material than the first layer.

25. A method of forming a medical lead comprising:
forming a RF shunted sleeve head;
coupling the RF shunted sleeve head to a lead body; and
preventing RF from affecting an operation of the medical lead.

26. A system for determining optimal materials to form a RF shunted sleeve head for an implantable medical lead comprising:
an electrochemical analyzer that includes a reference electrode, a tip electrode, and a counter electrode;
a container that includes a 0.1% saline sponge;
a RF shunted sleeve head of a lead coupled to the saline sponge;
a ring electrode of the lead disposed in a saline solution of the container;
and the reference electrode coupled to the saline sponge.
